(19) United States
(12) Patent Application Publication
(10) Pub. No.: US 2023/0355915 A1
(43) Pub. Date: Nov. 9, 2023

(54) DEVICE AND METHOD FOR CHANGING A VENTILATION MODE

(71) Applicant: Loewenstein Medical Technology S.A., Luxembourg (LU)

(72) Inventors: Benjamin ADAMETZ, Hamburg (DE); Marcel MEHNERT, Kremperheide (DE)

(21) Appl. No.: 18/246,070

(22) PCT Filed: Sep. 23, 2021
(86) PCT No.: PCT/EP2021/025361

§ 371 Date: Mar. 21, 2023

(30) Foreign Application Priority Data
Sep. 23, 2020 (DE) 102020124779.6

(51) Int. Cl. A61M 16/20; A61M 16/00; A61M 16/06

(52) U.S. Cl. CPC A61M 16/202; A61M 16/0003; A61M 16/0616; A61M 2205/3337; A61M 2205/3331; A61M 2016/0018

(57) ABSTRACT

Apparatus for supplying respiratory gas, comprising a respiratory gas source, a control unit, a storage device, a pressure sensor instrument and/or a flow sensor instrument, a replaceable respiratory gas tube, at least one connection fitting for the respiratory gas tube, a patient interface, and a patient valve, wherein the control unit activates a first ventilation mode for a first duration and in the process actuates the respiratory gas source to specify a changing respiratory gas parameter, and the control unit activates a further therapy mode for a second duration and in the process actuates the respiratory gas source to specify a respiratory gas parameter specific to the further therapy mode, the respiratory gas tube remaining on the apparatus when switching from the first ventilation mode to the further therapy mode, and the patient valve being switched for the further therapy mode by the control unit.

Ventilator apparatuses are used for the therapy of respiratory insufficiencies, in which context the ventilator apparatuses may be used in non-invasive and invasive ventilation, both in clinics and outside clinics.

For ventilation of a patient, a ventilator apparatus having an inspiratory branch for the inspiratory breathing gas flow and optionally having a branch for the expiratory breathing gas flow may in general be used. The branch for the expiratory breathing gas flow allows exhalation/expiration of a breathing gas by the patient, while the branch for the inspiratory breathing gas flow supplies the patient with breathing gas.

The ventilator apparatuses may be connected during operation to a tube system having a passive exhalation opening/leakage tube system or a tube system having an active exhalation valve/valve tube system.

In ventilator apparatuses known in the prior art, it is possible to change between ventilation with a leakage tube system and a valve tube system. For this purpose, however, it has hitherto been necessary to carry out conversion/installation of one or more components, for example a nonreturn valve, externally or internally to the apparatus.

A leakage tube system is a single tube system having a defined leakage opening, through which breathing gas can progressively escape during the ventilation in order to flush out carbon dioxide. A valve tube system is a single tube system having a switching valve for the expiration, or a double tube system in which exhaled breathing gas is returned to the ventilator apparatus for monitoring. Ventilator apparatuses therefore usually have at least two connection fittings, either for the double tube system or for the single tube system. Ventilator apparatuses additionally have pressure fittings, which are necessary for the use of single tube systems with a valve in order to supply a control pressure to the valve. In ventilator apparatuses known in the prior art, a suitable adapter therefore always needs to be used for the selected tube system, and pressure fittings may furthermore need to be closed or opened.

Before the patient tube system can be connected, the appropriate tube system adapter needs to be installed. The installation/conversion is however time-consuming and susceptible to error, and constitutes hindrance to use. A further disadvantage is that the therapy mode is always dependent on the tube system. For instance, a CPAP mode always requires a leakage tube system so that breathing gas can progressively escape in order to flush out carbon dioxide. A CPAP mode is currently not possible with a single tube system having a switching valve.

It is therefore an object of the invention to provide a device which makes it possible to use a ventilator apparatus both with a leakage tube system and with a valve tube system, without conversion measures or without adapters, and furthermore to make different therapy modes possible with the same tube system.

This object is achieved by an apparatus as claimed in claim 1. The dependent claims relate to developments and advantageous configurations. Further advantages and features may be found from the general description and the description of the exemplary embodiments.

Apparatus for breathing gas supply, comprising a breathing gas source, a control unit, a memory, a pressure sensor instrument and/or a flow sensor instrument, a replaceable breathing gas tube, at least one connection fitting for the breathing gas tube, a patient interface and a patient valve, wherein the control unit activates a first mode of ventilation for a first period of time, and in this case drives the breathing gas source in order to specify a varying breathing gas parameter for the ventilation, and wherein the control unit activates a further therapy mode for a second period of time, and in this case drives the breathing gas source in order to specify a breathing gas parameter specific for this therapy mode, characterized in that the breathing gas tube remains on the apparatus during the change from the first mode of ventilation to the further therapy mode, and the patient valve is switched for the further therapy mode by the control unit.

In one development, the apparatus is characterized in that the control unit activates a CPAP mode or an MPV mode or an HFT mode as a further therapy mode. Wherein the breathing gas tube may be a single tube-valve system.

In one development, the apparatus is characterized in that the control unit activates a further therapy mode, and in this case drives the breathing gas source in order to specify a constant breathing gas parameter independent of or dependent on the breathing phase.

In one development, the apparatus is also characterized in that the further therapy mode is a constant CPAP pressure, which is maintained independently of the breathing phase.

In one development, the apparatus is additionally characterized in that the valve is opened or closed as a function of the breathing phase.

In one development, the apparatus is characterized in that the valve is closed during the inspiration, and is driven in a controlled way, and temporarily opened in order to ensure exhalation, during the expiration.

In one development, the apparatus is characterized in that the patient breathing is identified by the control unit from the profile of the flow signal of the flow sensor instrument, and the valve is actuated as a function of the flow signal (as a trigger).

In one development, the apparatus is alternatively characterized in that limit values are stored or adjustable for the flow signal, the limit values being the trigger sensitivity.

In one development, the apparatus is for example characterized in that the control unit drives the breathing gas source in order to deliver breathing gas, in order to ensure maintenance of the CPAP pressure level during the switching processes of the valve.

In one development, the apparatus is characterized in that the control unit lowers the CPAP pressure at least temporarily when the patient breathing is identified by the control unit as expiration from the profile of the flow signal of the flow sensor instrument.

In one development, the apparatus is characterized in that the control unit raises the CPAP pressure at least temporarily (pursed lip breathing) when the patient breathing is identified by the control unit as expiration from the profile of the flow signal of the flow sensor instrument.

In one development, the apparatus is characterized in that the control unit may also specify the CPAP pressure at pressure values below hPa, since purging of CO from the exhaled air by the valve takes place reliably even at low pressures.

In one development, the apparatus is characterized in that the control unit activates a further therapy mode of CPAP in response to user selection or automatically, and in this case drives the breathing gas source in order to specify a CPAP pressure, the breathing gas tube remaining at the fitting on the apparatus during the change from the first mode of ventilation to the further therapy mode of CPAP, and the valve being closed during the inspiration, and being driven in a controlled way, and temporarily opened in order to ensure exhalation, during the expiration, the patient breathing being identified by the control unit from the profile of the flow signal of the flow sensor instrument, and the valve being actuated as a function of the flow signal (as a trigger), the breathing gas source being driven in order to ensure maintenance of the CPAP pressure level during the switching processes of the valve, the CPAP pressure also being specifiable at pressure values below hPa.

In one development, the apparatus is also characterized in that the further therapy mode is a constant flow (HFT), which is maintained independently of the breathing phase.

In one development, the apparatus is additionally characterized in that the control unit drives the breathing gas source in order to specify a substantially constant breathing gas flow and switches the patient valve into a permanently closed position.

In one development, the apparatus is also characterized in that the control unit is designed and configured to control the breathing gas source for the HFT mode in such a way that the patient flow decreases at the start during the expiration, while the mask pressure simultaneously increases.

In one development, the apparatus is furthermore characterized in that the apparatus additionally has, integrated or connected, at least one humidifier and/or an oxygen source and/or a nebulizer and/or at least one heater.

In one development, the apparatus is additionally characterized in that the control unit additionally activates the humidifier and the heater(s) in order to warm and humidify the breathing gas when the HFT mode is activated.

In one development, the apparatus is characterized in that the HFT mode drives the breathing gas source in order to specify a breathing gas flow in the range of 0-90 l/min, preferably 1-80 l/min, particularly preferably 2-60 l/min.

In one development, the apparatus is for example characterized in that the breathing gas tube has a patient valve and remains on the apparatus during the change from ventilation to HFT, and for the HFT the patient valve is closed by a control pressure being sent by the breathing gas source through the pressure tube to the patient valve and/or the humidifier and the heater are activated.

In one development, the apparatus is characterized in that a nose piece, having fittings which are inserted at least partially into the nostrils, is used as the patient interface for the HFT mode.

In one development, the apparatus is also characterized in that the control unit specifies the HFT mode with a consistent high flow of (warmed and humidified) breathing gas, which is applied via the patient interface into both nostrils of the patient in such a way that the flow flushes the nasal dead space, the patient interface in this case not sealing tightly with the nasal wall so that exhalation past the patient interface is possible, the setpoint flow during the HFT ventilation being kept substantially consistent at a pre-adjusted level, the valve being kept in a closed state 0 during the HFT ventilation since breathing gas is not intended to escape progressively from the valve but is continuously conveyed during inspiration and expiration to the patient interface (for this purpose, the control pressure for the valve is always kept above the mask pressure).

The apparatus is also characterized in that the further therapy mode is an MPV mode, which delivers a breathing gas volume or a breathing gas flow or a pressurized breathing gas to the patient for the inspiration as required.

In one development, the apparatus is characterized in that the control unit drives the breathing gas source in order to specify a breathing gas flow or breathing gas volume or a pressurized breathing gas for the inspiration and switches the patient valve into a permanently closed position.

In one development, the apparatus is characterized in that a breathing effort (inspiration attempt) of the patient is identified by the control unit from the profile of the flow signal or of the pressure signal, and the control unit drives the breathing gas source specifying a breathing gas flow or breathing gas volume when an inspiration attempt by the patient is identified from the profile of the flow signal or of the pressure signal.

In one development, the apparatus is characterized in that the pressure of the breathing assistance and the volume are adjustable.

In one development, the apparatus is characterized in that the pressure of the breathing assistance and the inspiration time Ti are adjustable.

In one development, the apparatus is also characterized in that limit values are stored or adjustable for the flow signal/or pressure signal, the limit values being the trigger sensitivity.

In one development, the apparatus is for example characterized in that the trigger sensitivity is adjustable in from 3 to 15 stages.

In one development, the apparatus is for example characterized in that a trigger blocking time (in the range of from 0.1 to 10 seconds) can be specified, breathing efforts by the patient, registered by sensing, being ignored by the control unit for the duration of the trigger blocking time.

In one development, the apparatus is for example characterized in that an MPV interface (mouth piece), which is configured in such a way that it is inserted at least partially into the mouth, is used as the patient interface for the MPV mode, the control unit being designed and configured to control the breathing gas source in such a way that the mask pressure has an increasing profile during the inspiration and the mask pressure falls more slowly than the setpoint pressure during the expiration.

In one development, the apparatus is for example characterized in that the valve is briefly opened for the expiration so that the pressure at the mouthpiece is lowered, and the valve is subsequently closed.

The apparatus is also characterized in that the further therapy mode is an MPV mode, which delivers breathing gas to the patient for the inspiration as required, the breathing gas pressure being adjustable and/or the breathing gas volume or the inspiration time Ti furthermore being specified, a mouth piece being used as the patient interface and breathing signals of the patient being registered by sensing when the mouthpiece is in the mouth as a pressure trigger and/or flow trigger in order to start the MPV ventilation, the patient being able to keep the mouthpiece in their mouth for the expiration, and the control unit then at least temporarily opening the valve for the expiration so that the patient can exhale their expiratory air through the fully or partially opened valve to the environment, the control unit activating the breathing gas source during the expiration in order to specify a flushing flow to assist flushing of exhaled air out from the tube.

The apparatus is also characterized in that the further therapy mode is an MPV mode, which temporarily sends a pressurized breathing gas flow to the patient, the apparatus consisting of the following: a breathing gas source, which temporarily sends a pressurized breathing gas flow to the airways; a patient interface in the form of a mouth piece, which can be at least partially inserted into an airway opening of the patient and removed again, the patient interface furthermore being configured so that the breathing gas flow is sent into the airways of the patient; at least one sensor, which generates output signals that, when the mouth piece is at least partially inserted into an airway opening of the patient, indicate that the patient is ready to receive a breathing gas flow through the mouth piece, the sensor being designed to establish whether the patient has performed breathing efforts; and at least one control unit, which analyzes the sensor signal in order to ascertain whether the patient has performed a breathing efforts that exceeds or falls below a limit value for initiating the sending of a temporary pressurized breathing gas flow, the control unit activating the breathing gas source when the limit value for initiating the sending of a temporary pressurized breathing gas flow is reached or exceeded, before specifying a temporary pressurized breathing gas flow, the control unit then activating this temporary pressurized breathing gas flow for the inspiration of the patient.

The apparatus is also characterized in that the control unit drives the breathing gas source in order to specify a breathing gas pressure in the range of 0-90 mbar, preferably 1-80 mbar, particularly preferably 2-60 mbar for the mode of ventilation.

The apparatus is also characterized in that the control unit drives the breathing gas source in order to specify a tidal volume for the mode of ventilation.

The apparatus is also characterized in that the apparatus has a compressed gas source and at least one pressure tube, which sends a control pressure to the patient valve.

The apparatus is also characterized in that the breathing gas source is the compressed gas source.

The apparatus is also characterized in that the breathing gas tube is a single tube system having a patient valve.

The apparatus is additionally characterized in that the breathing gas tube is a double tube system having a patient valve.

The apparatus is also characterized in that the breathing gas tube is a double tube system having an assigned patient valve, the patient valve being located next to the fitting in the apparatus housing.

The apparatus is additionally also characterized in that the patient valve is configured to be removable from a compartment of the housing, the patient valve having a membrane to which a control pressure can be applied in order to block or release a breathing gas flow through the valve.

The apparatus is also characterized in that the valve has a sealing membrane, to which a control pressure that opens or closes the valve is applied, the control pressure being generated by the breathing gas source and sent to the valve through a control tube.

The apparatus is also characterized in that the valve is electrically operated.

The apparatus is also characterized in that the control unit drives the breathing gas source in order to specify an inspiratory pressure having a specifiable pressure waveform.

The apparatus is also characterized in that the control unit drives the breathing gas source in order to specify an inspiratory pressure having two different inspiratory pressure levels.

The apparatus may also be characterized in that the control unit drives the breathing gas source in order to specify an expiratory pressure having a specifiable pressure waveform.

The apparatus may also be characterized in that the control unit drives the breathing gas source in order to specify an expiratory pressure having two different expiratory pressure levels, the pressure being raised starting from a low expiratory level to an increased expiratory level.

The apparatus may also be characterized in that the control unit drives the breathing gas source in order to specify an expiratory pressure and a ramped pressure increase to an inspiratory pressure level.

The apparatus may also be characterized in that the control unit drives the breathing gas source in order to specify an inspiratory pressure and a ramped pressure reduction to an expiratory pressure level.

The apparatus may also be characterized in that the control unit is configured to recognize breathing attempts from the pressure signal and/or from the flow signal of the pressure sensor instrument and/or of the flow sensor instrument.

The apparatus may also be characterized in that the patient interface is configured as a nasal cannula or flow cannula, as a nasal plug or mask or as a tracheostomy connection.

The apparatus may also be characterized in that a nasal cannula or flow cannula is used as a patient interface when the HFT mode is activated.

The apparatus may also be characterized in that a nasal plug, a mask or a tracheostomy connection is used as the patient interface when ventilation is activated.

The apparatus may be characterized in that the control unit drives the breathing gas source during the day in order to specify a breathing gas flow and during the night in order to specify a varying breathing gas pressure.

For all exemplary embodiments, the following apply alternatively or additionally:

For example, the control unit is designed and configured to control the breathing gas source in such a way that the specified control pressure is increased during the inspiration to a maximum pressure, which is reached before half of the duration of an inspiration or at the end of the inspiration. Preferably, the setpoint pressure is in this case initially exceeded slightly, in the range of 5-20%.

For example, the control unit is designed and configured to control the breathing gas source in such a way that the specified setpoint volume is increased during the inspiration to a maximum volume, which is reached before half of the duration of an inspiration. For example, the specified setpoint volume is in this case initially exceeded slightly, in the range of 2-15%.

For example, the control unit is designed and configured to control the breathing gas source in such a way that the specified control pressure is reduced during the expiration to a minimum pressure, which is reached before half of the duration of an expiration.

For example, the control unit is designed and configured to control the breathing gas source in such a way that the mask pressure has an increasing profile during the inspiration. For example, the mask pressure has a profile during the inspiration which is greatest at the end of the inspiration. Preferably, the setpoint pressure is in this case initially fallen below slightly, in the range of 5-20%, the setpoint pressure and mask pressure preferably being substantially equal at the end of the inspiration.

For example, the control unit is designed and configured to control the breathing gas source in such a way that the mask pressure initially increases during the expiration and then has a decreasing profile. Preferably, the mask pressure increases after the start of the expiration to a value which lies above the setpoint pressure and then falls—at least temporarily—to values which lie below the setpoint pressure.

Further preferably, in the MPV mode the valve is opened briefly for the subsequent expiration, so that the pressure at the mouth piece is reduced, and the valve is subsequently closed in order to detect the next breathing effort of the patient. For example, the valve may remain closed for the inspiration and be fully opened immediately after the end of the pressure specification, in order to allow the patient rapid expiration. Alternatively, the valve may be opened only partially after the end of the pressure specification, in order to allow the patient expiration but also to generate a therapeutically effective resistance during the exhalation, which keeps the small airways open for as long as possible and therefore allows comprehensive exhalation of CO2. Alternatively, the valve may be opened only partially after the end of the pressure specification so that the patient is allowed expiration against a dynamically regulated counter-pressure, as a function of the flow of the expiration, a therapeutically effective resistance being generated during the exhalation by the regulated partial opening or closing of the valve, which keeps the small airways open for as long as possible and therefore allows comprehensive exhalation of CO2.

In the high-flow mode (HFT mode), the apparatus conveys the adjusted flow. In the HFT mode, the apparatus is used as a flow source for high-flow therapy. A non-sealing patient interface for the nose is conventionally used as the patient interface.

Der MPV mode (Mouth Piece Ventilation mode) is a spontaneous mode in which the patient decides freely when they receive breathing assistance. A mouthpiece is conventionally used as the patient interface.

The CPAP mode (Continuous Positive Airway Pressure) is a spontaneous mode in which the apparatus applies a permanent overpressure. A mask is conventionally used as the patient interface.

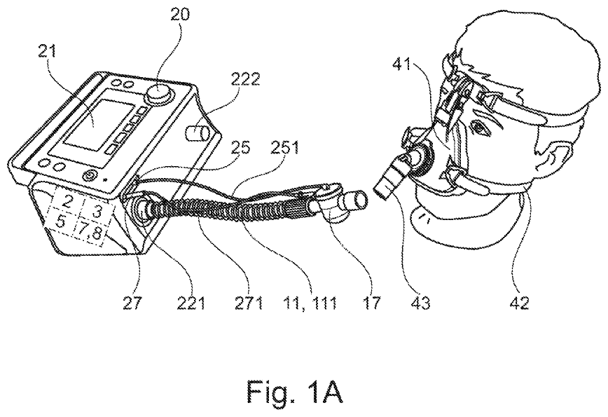

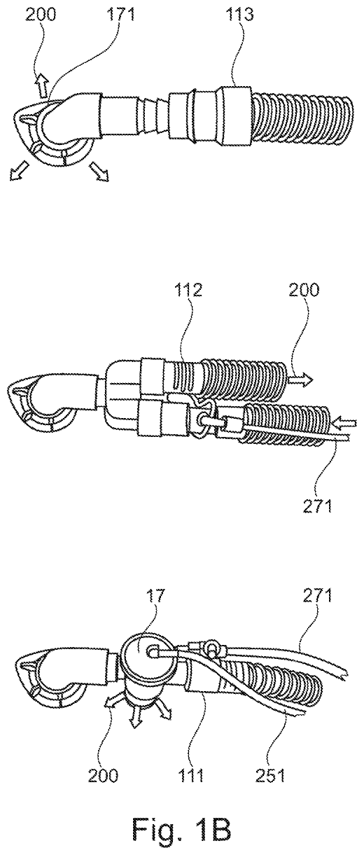

FIG. 1A shows the apparatus 1 according to the invention with a respiratory mask 41 as a patient interface 4. The mask is fastened on the head by strapping 42. The mask can be connected to the tube by means of a fitting 43.

The apparatus for breathing gas supply 1 comprises a breathing gas source 2, a control unit 3, a memory 5, a pressure sensor instrument 7 and/or a flow sensor instrument 8, a breathing gas tube 11 and a patient interface 4, which is configured here as a respiratory mask 41. The apparatus furthermore has an operating unit 20 and a display 21. The apparatus furthermore has two fittings 22 (221, 222) for the breathing gas tube 11. One fitting 221 is designed for the connection of the one breathing gas tube 11 in the form of a single-tube valve system 111. A leakage tube 113 may also be connected to this fitting.

Furthermore, the inspiratory branch of the double tube system 112 may be connected to this fitting 221. The other fitting 222 is used for connecting the expiratory branch of the double tube system 112.

FIG. 1B shows different tube systems. The apparatus may be used with a leakage tube 113 (top), a single tube-valve system 111 (bottom) or a double tube system 112 (middle).

In the case of a leakage tube 113, the exhaled air 200 containing CO2 is flushed continuously by means of an exhalation system 171.

In the single tube-valve system and the double tube system, the exhalation of the patient is controlled by means of a valve 17.

In the double tube system 112, the valve 17 is arranged in the apparatus. The exhaled air is sent via a partial tube to the exp. entry fitting 222 of the ventilator apparatus and is released from there into the environment through the valve 17. For this purpose, the valve opens with each expiration. The valve is closed with each inspiration. A pressure measuring tube 271 taps the pressure in the two tube system.

In the single tube-valve system 111, the valve 17 is arranged in or on the tube 11.

The valve 17 has for example three basic gas paths as well as an opening, which is provided with a sealing membrane. The gas paths are a closable expiratory gas path, an inspiratory gas path, which leads to the ventilator apparatus and through which inspiratory breathing gas flows, and a patient gas path, which leads to the patient interface. Flowing through the patient gas path is inspiratory breathing gas during the inspiration and expiratory breathing gas during the expiration. The expiratory gas path communicates with the opening, which can be fully closed or opened by means of the membrane.

In FIG. 1, the opening and the sealing membrane are covered by a closure cap. The pressure tube 251 debouches onto the closure cap. The pressure tube 251 supplies the control pressure to the membrane. The membrane then closes the opening that leads to the expiratory gas path.

The valve may be operated/controlled pneumatically. Whether arranged in the apparatus or on the tube, the valve receives for example a control pressure that opens or closes the valve. The valve has a sealing membrane, to which a control pressure that opens or closes the valve is applied, the control pressure being generated by the breathing gas source 2 and sent to the valve 17 through a control tube (not shown).

A pressure measuring tube 271 taps the pressure in the tube next to the valve. The control pressure is generated by the breathing gas source 2 and is sent to the valve through a control tube (not shown). In this case, for example, the control pressure is sent first to the internal valve 17, which is arranged next to the exp. entry fitting 222. From there, the pressure may also be sent to the second valve 17 (in the single tube system). An interrupter (not shown) frees or blocks the path to the single tube-valve system 111.

For the single tube system, a pressure fitting 25 at which the control pressure is applied is arranged on the apparatus housing. The control pressure is sent to the valve 17 through a pressure tube 251. The pressure fitting 25 may be closed in order to prevent a pressure loss when not being used.

A fitting 27 for a pressure measurement is also arranged next to the fitting 221. The apparatus contains a pressure sensor, which is pneumatically assigned to the fitting 27. A pressure measuring tube 271, which determines the pressure in the tube in the region before or after the valve 17 (in the flow direction), can be adapted to the fitting 27.

A fitting for a pressure measurement may also be arranged next to the fitting 222. The apparatus contains a pressure sensor, which is pneumatically assigned to the fitting. A pressure measuring tube 271, which determines the pressure in the exp. tube or in the region before or after the valve (in the flow direction), can be adapted to the fitting. This pressure measurement is expedient in order to determine, and optionally regulate, compliance with the pressure specification for the expiration.

According to the invention, the valve 17 may be controlled electronically. Then, for example, it is supplied with energy via a cable connection from the apparatus, or by means of a battery which is arranged next to the valve.

Alternatively, the valve may be electrically operated/controlled, the membrane being for example moved against the opening by an electrically operated actuator.

Alternatively, the valve may for example be electrically operated/controlled as an axial voice coil actuator (VCA). The latter consists of a permanent magnet in a movable tubular coil of wire, which is located in a ferromagnetic cylinder. When current flows through the coil, it becomes magnetized and repels the magnet. In this way, a movement inward and outward as well as forward and backward is generated. Further advantages of linear VCA motors are their bidirectionality and the presence of permanent magnets and magnetic holding coils. They make it possible to remain at one end of the travel when the current supply is interrupted—for example in order to ensure that valves remain open or closed in the event of a power outage. VCAs accelerate uniformly and rapidly within the travel, with almost no hysteresis. The valve in the tube and/or the valve in the apparatus may be electrically operated/controlled.

In this configuration, the apparatus is designed for example for the mode of ventilation 6 and the mode of CPAP 61.

For example, the control unit 3 initially activates the first mode of ventilation 6, while driving the breathing gas source 2 in order to specify a breathing phase-dependent and varying breathing gas parameter, namely breathing gas pressure or breathing gas volume, for the ventilation. For example, the control unit specifies a higher breathing gas pressure (IPBP) as a setpoint value for the inspiration than for the expiration (EPAP).

The control unit 3 activates a further therapy mode of CPAP 61 in response to user selection or automatically. The breathing gas source 2 is in this case driven in order to specify a constant CPAP pressure. The CPAP pressure is preferably maintained independently of the breathing phase.

During the change from the first mode of ventilation 6 to the further therapy mode of CPAP 61, the breathing gas tube 11 remains on the apparatus 1. The patient valve 17 is in this case switched by the control unit in order to specify the further therapy mode.

The valve 17 is closed during the inspiration and is driven in a controlled way, and temporarily opened in order to ensure exhalation, during the expiration.

For this purpose, the patient breathing is identified by the control unit 3 from the profile of the flow signal of the flow sensor instrument 8, and the valve 17 is actuated as a function of the flow signal (as a trigger).

Limit values are stored or adjustable for the flow signal. The limit values represent the change between inspiration and expiration, and are therefore used as trigger signals for the driving of the valve 17. According to the invention, a pressure trigger or a combination of both trigger options are also possible. In the case of the pressure trigger, the inspiration is recognized by the control unit by the pressure decreasing slightly, and the expiration is recognized by the pressure rising slightly. Limit values are stored or adjustable for the pressure signal. The limit values represent the change between inspiration and expiration, and are therefore used as trigger signals for the driving of the valve 17.

The control unit 3 drives the breathing gas source in order to ensure maintenance of the CPAP pressure level during the switching processes of the valve 17.

The control unit 3 lowers the CPAP pressure at least temporarily for example, when the patient breathing is identified by the control unit 3 as expiration from the profile of the flow signal of the flow sensor instrument 8. This makes exhalation more comfortable for the patient.

Alternatively, for example, the control unit 3 raises the CPAP pressure at least temporarily (pursed lip breathing) when the patient breathing is identified by the control unit 3 as expiration from the profile of the flow signal of the flow sensor instrument 8. Closed lung regions may be opened up by the increased pressure, and the expiration can then take place fully.

The control unit 3 may also specify the CPAP pressure at pressure values below 4 hPa since, according to the degree of opening of the valve 17, purging of CO2 from the exhaled air by the valve takes place reliably even at low pressures. In this case, the valve is opened at least temporarily to such an extent that breathing gas (consisting of breathing gas from the exhalation and fresh exhaled gas) can flow away. The functionality of the valve then resembles that of a leakage system.

The apparatus has a user interface which is designed and configured as an operating and display element, the operating and display element being formed in a face of the housing of the device, the operating and display element being configured with such a large surface that it occupies more than 45% or preferably more than 50% of the area of the housing, in particular the top wall.

In another development, the apparatus has a user interface which is designed and configured as an operating and display element. In general, the operating and display element is configured as a GUI. In general, the GUI is configured as a touchscreen. Optionally, the operating and display element comprises haptic operating elements. A haptic operating element may be arranged on the top wall or on a side wall of the device. Optionally, the operating and display element may be designed to emit an acoustic or haptic confirmation in the event of an adjustment.

In one configuration, the operating and display element is formed in a face of the housing of the device, in particular the top wall, the operating and display element being designed to reproduce a representation, the alignment of the representation being carried out as a function of the selected bottom wall. The operating and display element is therefore designed to change an orientation of the alignment of the display when the alignment of the device is changed according to a selected bottom wall. The apparatus is in general designed to change/adapt the alignment of the display automatically according to the alignment of the device.

In another development, the operating and display element is designed to register an ambient brightness and to carry out a change of an optical display of the operating and display element on the basis of the registered ambient brightness, the operating and display element being designed to change a color intensity or change from a color display to a black and white display when a bright ambient brightness is registered. This offers the advantage that the visibility of the display of the operating and display element may be improved in the event of a bright ambient brightness.

The operating and display element is designed to increase the color intensity of the display according to a registered brightness of the ambient brightness, or to change to a black and white display in the event of a strong brightness. By omitting a color representation, a better display may be achieved by the contrast increase in the event of a strong ambient brightness. For example, the operating and display element may be designed to change from a color representation to a black and white representation. This is advantageous in particular for mobile carrying of the device, particularly outdoors. The operating and display element is designed to be dimmable according to a state of charge of the rechargeable battery.

In one configuration, the apparatus comprises a digital interface which is designed to transmit registered parameters, measurement values and information to a server or an external terminal, and to receive data and information via the interface. Optionally, the apparatus is designed to store, analyze and/or assess the registered values and/or information of the measurement segment. The apparatus may be coupled with and exchange data with a cough apparatus or another ventilator apparatus or a patient monitor via the interface.

Optionally, the apparatus is designed to transmit the registered, analyzed and/or assessed measurement values/parameters to an external server. The transmission may in this case be designed to be time-controlled, manually initiated (for example initiated on a home therapy apparatus or on a server), event-controlled (for example when particular critical states are recognized by the therapy apparatus) or as a continuous transmission, at least during an ongoing therapy.

Transmission of measurement values, parameters and information may take place every 2 hours to 7 days, in particular every 1 to 3 days. In one embodiment, the transmission takes place at least once per day/per 24 hours. Optionally, the interface may be designed to transmit measurement values, information or parameters hourly as a summary, or to transmit the measurement values in real time. Optionally, a transmission cycle is freely selectable by the user and/or by a caretaker. The interface of the ventilator apparatus may be designed to carry out the transmission automatically, optionally repeatedly or continuously, according to one or more time intervals that are permanently input and/or freely input.

In the event of a failure of a data connection, the memory unit of the device may be designed to store the measurement values and/or the information for at least one day, the interface of the device being designed to transmit the measurement values to an external server or a terminal as soon as a data connection has been re-established.

Optionally, the apparatus is designed to include information the values manually input by the user and/or by the carer via the operating and display element in the evaluation of measurement values.

In another configuration, the apparatus comprises an alarm unit that has a loudspeaker and is designed to emit an alarm when incidents are recognized, the apparatus comprising at least one microphone that is designed to monitor an alarm emitted by the alarm unit. This offers an additional safety function for correct use of the device for ventilation.

In one configuration, the apparatus is designed to be combinable with further devices. Optionally, the apparatus has a connection for a nebulizer, the apparatus being designed to control the nebulizer, when it is connected, by means of the apparatus. The apparatus is optionally designed to register a response from the nebulizer and take it into account in the control of the nebulizer.

The apparatus comprises connections for a server, a patient management system, a cough device and a sleep laboratory infrastructure. The apparatus furthermore comprises a cloud function, the apparatus being designed to transmit data via an interface to a cloud, or a connection for a GSM module. In one development, the apparatus comprises a connection for a nurse call module. The apparatus furthermore comprises at least one SpO2 and/or CO2 connection.

The apparatus has, for example, the following operating states:

On: The therapy is ongoing. Apparatus and therapy adjustments are possible.
Standby: The fan is off and the therapy is not ongoing. The apparatus is, however, immediately ready for operation. Apparatus and therapy adjustments are possible.
Off: The apparatus is turned off. Adjustments are not possible and the display remains dark.

The ventilator apparatus is intended for continuous or intermittent breathing assistance for the care of persons who need to be mechanically ventilated. The ventilator apparatus is intended in particular for children and adults having a minimum title volume of 30 ml. The apparatus is suitable for use in the home setting, in care facilities and in hospitals, as well as for mobile applications, for example in a wheelchair or on a gurney. It may be employed for invasive and non-invasive ventilation. The apparatus is also intended for use as a ventilator apparatus during transport or in intensive care.

The apparatus may be used with both non-invasive and invasive patient interfaces (ventilatory accesses). A fan takes in the ambient air through a filter and conveys it at the therapy pressure through the breathing tube and the ventilatory access to the patient. On the basis of the registered signals of the pressure and flow sensors, the fan is controlled according to the breathing phases. The operating interface is used for displaying and adjusting the available parameters and alarms. The apparatus may be used both with a leakage tube and a single tube-valve system, or a double tube system. In the case of a leakage tube, exhaled air containing CO2 is flushed continuously by means of an exhalation system. In the single tube-valve system and the double tube system, the exhalation of the patient is controlled by means of a valve.

In the high-flow mode (HFT mode), the apparatus conveys the adjusted flow to an external HFT-compatible humidifier. The latter conditions the breathing gas in respect of temperature and air humidity. The patient connection is carried out by means of an HFT-compatible accessory. The HFT mode (when available) and the MPV mode are specific modes since a fixed and/or sealed connection is not established between the corresponding accesses and the airways of the patient, and so some provisions, such as the recognition of a disconnection, are not used. Oxygen may be introduced through the oxygen entry point. With an integrated FiO2 sensor, the FiO2 concentration output by the apparatus may be measured as required. It is also possible to feed in an external SpO2 measurement. The power supply is carried out through an external power supply unit. The apparatus has a built-in battery and can therefore continue to be operated without interruption in the event of a power outage. In addition, a maximum of two external batteries may be connected in order to operate the apparatus. The therapy data are stored in the apparatus and may additionally be loaded onto a USB-C stick and evaluated by means of PC software.

The breathing gas drive may be a fan, a valve, an oxygen source (high-pressure) or a compressed air source (high-pressure) or a combination thereof. The breathing gas drive is, for example, arranged freely suspended in the apparatus via at least two, in particular three, anchoring points.

The control unit comprises in general at least one memory unit and an evaluation unit. The memory unit is designed to store measurement values, information and/or parameters and provide them for evaluations by the evaluation unit. The evaluation unit is designed to compare the measurement values, information and/or parameters with one another or with external data. The control unit is designed to receive, store and analyze data from components of the device, in particular a measuring unit of the flow measurement segment. Optionally, the control unit is designed to transmit data, measurement values, information and/or parameters to a digital interface of the device.

The apparatus is also designed in particular for use in pediatric ventilation. The device comprises stored ventilation modes. In particular, the apparatus comprises at least one high-flow mode and at least one PEEP control mode. In general, the control unit of the device is designed to adjust the ventilation modes, frequencies, triggers and flows of the device.

The apparatus may be used with a leakage tube, a single tube-valve system or a double tube system. In the case of a leakage tube, exhaled air containing CO2 is flushed continuously by means of an exhalation system.

In the single tube-valve system and the double tube system, the exhalation of the patient is controlled by means of a valve.

In the double tube system, the valve is arranged in the apparatus. The exhaled air is sent via a partial tube to the exp. entry fitting of the ventilator apparatus and is released from there into the environment through the valve. For this purpose, the valve opens with each expiration. The valve is closed with each inspiration.

In the single tube-valve system, the valve is arranged in or on the tube. Whether arranged internally or externally, the valve always receives a control pressure that opens or closes the valve.

The control pressure is generated by the breathing gas source and is sent to the valve through a control tube. In this case, the control pressure is sent first to the internal valve. From there, the pressure may be sent to the second valve (in the single tube system). An interrupter frees or blocks the path.

For the single tube system, a pressure fitting at which the control pressure is applied is arranged on the apparatus housing. The control pressure is sent to the valve through a pressure tube.

The invention described above has the advantage overall that ventilation of a patient is made possible, while the patient's mobility can be maintained. For example, the device may be fitted on a wheelchair. The device furthermore comprises, for example, an aspiration function and a cough mode. The apparatus may be adapted to various tube systems without conversion of the connection region of the tube system on the ventilator apparatus being carried out.

In the high-flow mode (HFT mode), the apparatus conveys the adjusted flow to an external HFT-compatible humidifier. The latter conditions the breathing gas in respect of temperature and air humidity. The patient connection is carried out by means of an HFT-compatible accessory. In the HFT mode, the apparatus is used as a flow source for the high-flow therapy. 5 l/min to 60 l/min (adult), 5 l/min to 25 l/min (child)

The MPV mode (Mouth Piece Ventilation mode) is a spontaneous mode in which the patient decides freely when they receive breathing assistance. Distinction is made between pressure specification and volume specification.

For pressure specification: the Inspiratory Positive Airway Pressure (IPAP) may be adjusted in the range of 4-50 hPa/mbar/cmH2O when using the leakage tube system, or in the range of 4-60 hPa/mbar/cmH2O when using the single or double tube-valve system.

The CPAP mode (Continuous Positive Airway Pressure) is a spontaneous mode in which the ventilator apparatus applies a permanent overpressure. The CPAP mode may be applied as an invasive ventilation method, that is to say through a tube or a tracheal cannula, and alternatively also as non-invasive ventilation (NIV), that is to say through a mask (for example a mouth and nose mask, nose mask, face mask, oral mask or helmet. The CPAP pressure may be adjusted in the range of 0-50 hPa/mbar/cmH2O.

Inspiration time (Ti) may be adjusted for spontaneous breathing. In the range of from 0.2 seconds to 4 seconds for children and from 0.5 seconds to 5 seconds for adults. The inspiration is ended at the latest after Ti has elapsed.

Mandatory breath: Ti is fixed.

The trigger sensitivity may be adjusted in 10 stages. Likewise, a trigger blocking time may be adjusted. Inspiratory trigger signals are ignored in the adjusted period of time, which lies in the range of from 0.2 s to 5 s.

For volume specification: the Inspiratory Positive Airway Pressure (IPAP) may be adjusted in the range of 4-50 hPa/mbar/cmH2O when using the leakage tube system, or in the range of 4-60 hPa/mbar/cmH2O when using the single or double tube-valve system.

The delivered volume (Vt) may be adjusted. In the range of from 30 ml to 400 ml for children and from 100 ml to 3000 ml for adults.

The trigger sensitivity may be adjusted in 10 stages. Likewise, a trigger blocking time may be adjusted. Inspiratory trigger signals are ignored in the adjusted period of time, which lies in the range of from 0.2 s to 5 s.

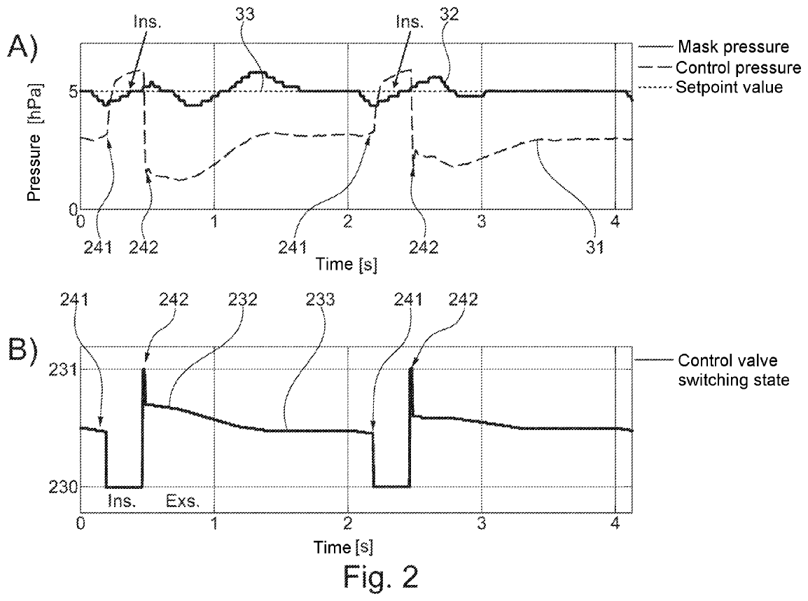

FIG. 2 shows the specification for CPAP therapy/CPAP mode.

In FIG. 2A, the pressure is shown above. The control pressure 31 as a specification value of the control unit 3 for the valve 17, the mask pressure 32 (or in the scope of the invention generally the pressure 32 in the patient interface), which is determined by the pressure sensor 7, and the setpoint pressure 33 as a specification value of the control unit 3 for the breathing gas source are plotted. The mask pressure 32 is the resulting pressure which is therapeutically effectively applied in the patient interface for the patient. The mask pressure 32 is the resulting pressure which is determined from the breathing activity of the patient and/or the control pressure 31 and/or the switching state of the valve 17.

The pressure is indicated in the unit hPa. A time axis is indicated below in seconds. FIG. 2 shows overall a recording over 4 seconds, which here by way of example represents rapid breathing.

In FIG. 2B, the switching state 23 of the valve 17 and the breathing phase 24 (241, 242) of the patient are shown.

From a comparison of FIGS. 2B and 2A, it can be seen that the change from an inspiration 241 to an expiration 242 takes place in the time range of the first second. The inspiration takes place between the instants 241 and 242. The expiration takes place between the instants 242 and 241.

It can be seen in FIG. 2A that the control unit briefly opens 231 the valve 17 for the change from an inspiration 241 to an expiration 242. For this purpose, the valve 17 is switched from the closed state 230 into the fully opened state 231.

It can be seen in FIG. 2A that the control pressure 31 for the valve is lowered at this instant from a maximum value to a minimum value. The control pressure 31 in this case falls below the mask pressure 31. When the mask pressure 32 is fallen below, the valve 17 opens and the exhaled air can escape.

At the same time, with the start of the expiration the mask pressure 32 initially rises. It can be seen in FIG. 2B that the switching state of the valve is fully opened 231 only for a very short time (less than 0.5 seconds, preferably less than 0.25 seconds). Immediately after the complete opening, the valve partially closes. The valve is switched into a partially opened state 232 which approximately lies in the range of 50-80%, preferably 60-75% of complete opening. The valve is subsequently switched for a third time segment into a half-opened state 233 in which it is 40-60%, preferably 45-55%, opened. This half-opened state 233 lasts for about half of the expiration.

The fully opened 231 time segment lasts less than 10% of the duration of the expiration. Preferably, the fully opened 231 time segment lasts less than 5% of the duration of the expiration.

The second segment with the partially opened state 232 lasts in the range of 25%-50% of the expiration. In the partially opened state 232, the switching state is for example modified further in the direction of the modified half-opened state 233.

The valve remains in the half-opened state 233 for a duration in the range of 25%-55% of the expiration.

With the end of the expiration 241, the valve is switched into the fully closed state 230. For the duration of the inspiration, the valve remains in the fully closed state 230.

It can be seen from FIG. 2A that the control pressure 32 therefore lies above the setpoint pressure for the duration of the inspiration (Ins.). It can also be seen from FIG. 2A that the control pressure is brought to a minimum value at the start of the expiration in order to open the valve fully. This corresponds to the first time segment. For the second time segment, the control pressure is increased minimally. For the third time segment, the control pressure is increased some more. The control pressure remains at the increased level for the third time segment. With the end of the expiration, the control pressure is increased to a maximum value. The effect of this is that the valve is fully closed, which may be seen in FIG. 2 from the fact that the switching state of the valve is closed 230 for example in the time range between seconds two and three. It can be seen in FIG. 2A that with the start of the inspiration, the mask pressure decreases and lies below the setpoint pressure. The mask pressure increases again after the start of the inspiration. After about half of the inspiration time, the mask pressure again reaches the setpoint value. The mask pressure subsequently exceeds the setpoint value and reaches a maximum at the start of the next expiration. For the subsequent expiration, it may be seen from an overview of FIG. 2 that the switching states and the pressure profiles for the expiration are almost identical to the switching states and the pressure profiles of the first expiration.

For example, the control unit is designed and configured to control the breathing gas source in such a way that the specified control pressure is increased during the inspiration to a maximum pressure, which is reached before half of the duration of an inspiration or at the end of the inspiration. Preferably, the setpoint pressure is in this case initially exceeded slightly, in the range of 5-20%.

For example, the control unit is designed and configured to control the breathing gas source in such a way that the specified setpoint volume is increased during the inspiration to a maximum volume, which is reached before half of the duration of an inspiration. For example, the specified setpoint volume is in this case initially exceeded slightly, in the range of 2-15%.

For example, the control unit is designed and configured to control the breathing gas source in such a way that the specified control pressure is reduced during the expiration to a minimum pressure, which is reached before half of the duration of an expiration.

For example, the control unit is designed and configured to control the breathing gas source in such a way that the mask pressure has an increasing profile during the inspiration. For example, the mask pressure during the inspiration has a profile which is greatest at the end of the inspiration. Preferably, the setpoint pressure is in this case initially fallen below slightly, in the range of 5-20%, the setpoint pressure and mask pressure preferably being substantially equal at the end of the inspiration.

For example, the control unit is designed and configured to control the breathing gas source in such a way that the mask pressure initially increases and then has a decreasing profile during the expiration. Preferably, the mask pressure increases after the start of the expiration to a value which lies above the setpoint pressure and then falls—at least temporarily—to values which lie below the setpoint pressure.

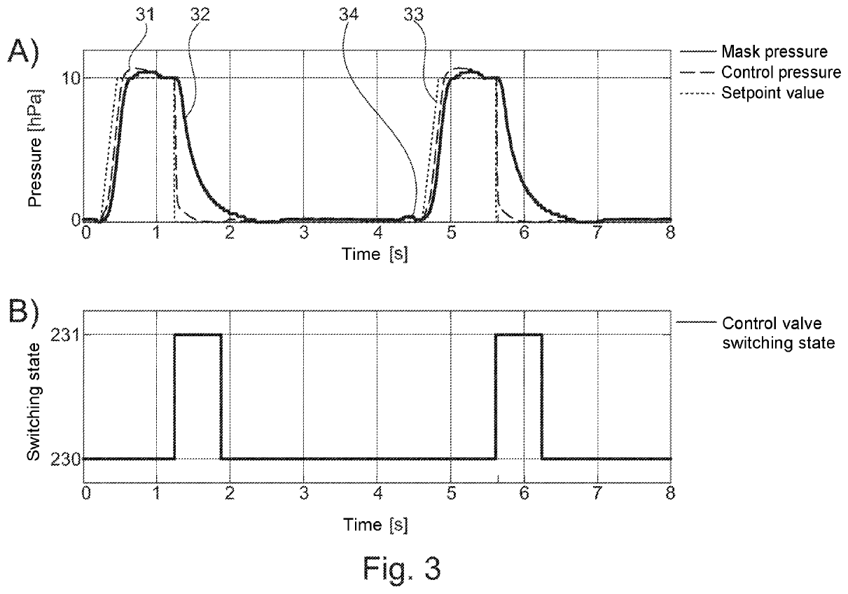

FIG. 3 shows the specification for the MPV therapy. The MPV mode (Mouth Piece Ventilation mode) is a spontaneous mode in which the patient decides freely when they receive breathing assistance. A mouth piece is used instead of a mask as the patient interface.

In FIG. 3A, the pressure is shown above. The control pressure 31 as a specification value of the control unit 3 for the valve 17, the mask pressure 32 (or mouth piece pressure), which is determined by the pressure sensor 7, and the setpoint pressure 33 as a specification value of the control unit 3 for the breathing gas source are plotted. The pressure is indicated in the unit hPa. A time axis is indicated below in seconds. FIG. 3 shows overall a recording over 8 seconds.

FIG. 3B shows the switching state 23 of the valve 17 in the course of two inspirations and expirations.

In FIG. 3, the pressure profile and the switching state of the valve for the MPV ventilation may be seen. In FIG. 3A, the control pressure 31 of the valve, the mask pressure 32 and the setpoint pressure 33 are plotted. It may be seen from the profile of the mask pressure that the mask pressure rises briefly in the time range between seconds four and five. This short pressure increase 34 occurs when the patient takes the mouth piece in their mouth. In the MPV mode, a low basic flow may be delivered continuously in order to identify when the patient takes the mouth piece in their mouth. Reception in the mouth then leads to a short pressure increase 34. This breathing signal of the patient may be evaluated by the control as a trigger 34 in order to initiate the MPV ventilation.

Alternatively or in addition, a flow trigger may be used in order to identify a breathing signal of the patient. In the MPV mode, a low basic flow may be delivered continuously in order to identify when the patient takes the mouth piece in their mouth. Reception in the mouth then leads to a short pressure decrease. Even without a basic flow, by means of the flow signal it is possible to identify when the patient takes the mouth piece in their mouth. When the patient has the mouth piece in their mouth and has inhaled or exhaled slightly, this breathing signal of the patient may also be used as a trigger in order to start the MPV ventilation.

In FIG. 3A, in response to the trigger signal, the control unit increases the pressure setpoint value 33 to ten hectopascals. For this purpose, the control unit drives the breathing gas source in order to specify this setpoint pressure and therefore delivers breathing gas to the patient for their inspiration. The mask pressure 32 increases rapidly according to the setpoint pressure 33 and exceeds the setpoint pressure slightly. A specification for the inspiration therefore takes place, which occurs in the time range between seconds zero and two, as well as in the time range of between seconds four and six.

From a comparison with FIG. 3B, it may be seen that the valve is still fully closed 230 in this period of time of the inspiration. It may be seen in FIG. 3A that the setpoint pressure 33 for the pressure is maintained for about 1 second. The mask pressure is likewise at the maximum value for about 1 second; the mask pressure follows the setpoint pressure. The control pressure 31 of the valve increases shortly after the setpoint pressure rises, in order to keep the valve 17 closed during the inspiration.

This means that a breathing gas flow or a breathing gas volume is conveyed to the patient for about 1 second. The patient uses this for an inspiration.

For the expiration, the patient may remove the mouthpiece from their mouth and then exhale. The patient may, however, also leave the mouthpiece in their mouth. If the patient leaves the mouthpiece in their mouth, the control unit switches the valve 17 for the expiration, after the inspiration, into the fully opened state 231. This may be seen in FIG. 3B in the time range between seconds one and two and seconds 5.5 and 6.5. At the same time, the setpoint value of the pressure is switched back to 0 hPa, as may be seen from FIG. 3A. Accordingly, the mask pressure decreases steeply. And the control pressure 31 of the valve likewise decreases in order to open the control valve fully.

The fully opened state 231 of the valve is maintained for about one half of a second to one second. The patient may then carry out their expiration with the mouth piece in their mouth. They opened valve in this case offers the advantage that the patient can exhale their expiratory air through the fully opened valve to the environment. At the same time, the control unit conveys a small flushing flow of breathing gas to the patient during the expiration. This flushing flow can escape through the opened valve 17. The effect of this is that carbon dioxide possibly contained in the tube is purged from the valve. A breathing gas low in CO2 is correspondingly again available to the patient for the subsequent inhalation or expiration.

In the MPV mode, the apparatus alternatively or additionally offers breathing assistance by temporarily sending a pressurized breathing gas flow to the patient, the apparatus consisting for example of the following: a breathing gas source, which temporarily sends a pressurized breathing gas flow to the airways;

a patient interface 4 in the form of a mouth piece, which can be at least partially inserted into an airway opening of the patient and removed again, the patient interface 4 furthermore being configured so that the breathing gas flow is sent into the airways of the patient;
at least one sensor, which generates output signals that indicate when the mouth piece is at least partially inserted into an airway opening of the patient and therefore to establish that the patient is ready to receive a breathing gas flow through the mouth piece, the sensor being designed to establish whether the patient has performed breathing efforts; and
at least one control unit, which analyzes the sensor signal in order to ascertain whether the patient has performed a breathing efforts that exceeds or falls below a limit value for initiating the sending of a temporary pressurized breathing gas flow, the control unit activating the breathing gas source when the limit value for initiating the sending of a temporary pressurized breathing gas flow is reached or exceeded, the specifying a temporary pressurized breathing gas flow, the control unit then activating this temporary pressurized breathing gas flow for the duration of an inspiration or for a fraction of the duration of the inspiration of the patient.

Preferably, the pressure and/or the duration are in this case adjustable.

In the MPVp mode according to the invention, the pressure of the breathing assistance and the inspiration time Ti are adjustable.

| | ADJUSTABLE | |
|---|---|---|
| PARAMETER | VALUES | DESCRIPTION |
| IPAP | 4-50 hPa/mbar/cmH2O | Here, you adjust the inspiratory |
| | (leakage tube system) | positive airway pressure. |
| | 4-60 hPa/mbar/cmH2O | |
| | (single or double tube- | |
| | valve system) | |
| Tᵢ | 0.2 s-4 s (child) | Here, you adjust the inspiration time. |
| | 0.5 s-4 s (adult) | Spontaneous breathing: the inspiration |
| | | is ended at the latest after Tᵢ |
| | | has elapsed. |
| | | Mandatory breath: Tᵢ is fixed. |
| Inspiratory | 1-10 | Here, you adjust the trigger sensitivity: |
| trigger | | 1: very sensitive |
| | | 10: not very sensitive |
| Trigger blocking | 0.2 s-5 s | Inspiratory trigger signals are ignored |
| time | | in the adjusted period of time |

In the MPV mode, alternatively or in addition, the apparatus offers breathing assistance by temporarily sending a pressurized breathing gas flow to the patient, the apparatus consisting for example of the following: a breathing gas source, which sends a breathing gas flow to the airways for a defined duration; a patient interface 4 in the form of a mouth piece, which can be at least partially inserted into an airway opening of the patient and removed again, the patient interface 4 furthermore being configured so that the breathing gas flow is sent into the airways of the patient;

at least one sensor, which generates output signals that indicate when the mouthpiece is at least partially inserted into an airway opening of the patient and therefore to establish that the patient is ready to receive a breathing gas flow through the mouth piece, the sensor being designed to establish whether the patient has performed breathing efforts; and at least one control unit, which analyzes the sensor signal in order to ascertain whether the patient has performed a breathing efforts that exceeds or falls below a limit value for initiating the sending of the temporary breathing gas flow, the control unit activating the breathing gas source when the limit value for initiating the sending of the temporary breathing gas flow is reached or exceeded, the specifying the temporary breathing gas flow, the control unit then activating this temporary breathing gas flow for the defined duration.

As an alternative preference, the pressure of the breathing assistance and the volume are adjustable.

In the MPVv mode, the pressure of the breathing assistance and the volume are adjustable.

| | ADJUSTABLE | |
|---|---|---|
| PARAMETER | VALUES | DESCRIPTION |
| IPAP | 4-50 hPa/mbar/cmH2O | Here, you adjust the inspiratory |
| | (leakage tube system) | positive airway pressure |
| | 4-60 hPa/mbar/cmH2O | |
| | (single or double tube- | |
| | valve system) | |
| Volume | 30 ml-400 ml (child) | Here, you adjust the delivered volume |
| | 100 ml-3000 ml (adult) | (Vₜ). |
| Inspiratory | 1-10 | Here, you adjust the trigger sensitivity: |
| trigger | | 1: very sensitive |
| | | 10: not very sensitive |
| Trigger blocking | 0.2 s-5 s | Inspiratory trigger signals are ignored |
| time | | in the adjusted period of time |

In the MPV mode, alternatively or in addition, the apparatus offers breathing assistance by temporarily sending a breathing gas volume to the patient, the apparatus consisting for example of the following: a breathing gas source, which sends a defined breathing gas volume to the airways for a; a patient interface 4 in the form of a mouth piece, which can be at least partially inserted into an airway opening of the patient and removed again, the patient interface 4 furthermore being configured so that the breathing gas volume is sent into the airways of the patient;

at least one sensor, which generates output signals that indicate when the mouth piece is at least partially inserted into an airway opening of the patient and therefore to establish that the patient is ready to receive a breathing gas flow through the mouth piece, the sensor being designed to establish whether the patient has performed breathing efforts; and
at least one control unit, which analyzes the sensor signal in order to ascertain whether the patient has performed a breathing efforts that exceeds or falls below a limit value for initiating the sending of the breathing gas volume, the control unit activating the breathing gas source when the limit value for initiating the sending of the breathing gas volume is reached or exceeded, the specifying the breathing gas volume, the control unit then activating the breathing gas source in order to convey the breathing gas volume.

For example, the control unit is so designed and configured to control the breathing gas source in such a way that the specified setpoint pressure is increased during the inspiration to a maximum pressure, which is reached before half of the duration of an inspiration. Preferably, the setpoint pressure is in this case initially exceeded slightly, in the range of 5-15%.

For example, the control unit is so designed and configured to control the breathing gas source in such a way that the specified setpoint volume is increased during the inspiration to a maximum volume, which is reached before half of the duration of an inspiration. For example, the specified setpoint volume is in this case initially exceeded slightly, in the range of 2-15%.

For example, the control unit is designed and configured to control the breathing gas source in such a way that the specified control pressure is increased during the inspiration to a maximum pressure which is reached before half of the duration of an inspiration or at the end of the inspiration. Preferably, the setpoint pressure is in this case initially exceeded slightly, in the range of 5-20%.

For example, the control unit is designed and configured to control the breathing gas source in such a way that the specified setpoint volume is increased during the inspiration to a maximum volume, which is reached before half of the duration of an inspiration. For example, the specified setpoint volume is in this case initially exceeded slightly, in the range of 2-15%.

For example, the control unit is designed and configured to control the breathing gas source in such a way that the specified control pressure is reduced during the expiration to a minimum pressure, which is reached before half of the duration of an expiration.

For example, the control unit is designed and configured to control the breathing gas source in such a way that the mask pressure has an increasing profile during the inspiration. For example, the mask pressure has a profile during the inspiration which is greatest in the middle of the inspiration. Preferably, the setpoint pressure is in this case exceeded slightly, in the range of 5-20%, the setpoint pressure and mask pressure preferably being substantially equal at the end of the inspiration.

For example, the control unit is designed and configured to control the breathing gas source in such a way that the mask pressure falls more slowly than the setpoint pressure during the expiration. Preferably, the mask pressure does not fall to a value that lies in the range of the setpoint pressure until after the end of the expiration.

Further preferably, the valve 17 is opened briefly for the subsequent expiration, so that the pressure at the mouthpiece is reduced, and the valve is subsequently closed in order to detect the next breathing effort of the patient. For example, the valve may remain closed for the inspiration and be fully opened immediately after the end of the pressure specification, in order to allow the patient rapid expiration. Alternatively, the valve may be opened only partially after the end of the pressure specification, in order to allow the patient expiration but also to generate a therapeutically effective resistance during the exhalation, which keeps the small airways open for as long as possible and therefore allows comprehensive exhalation of CO2. Alternatively, the valve may be opened only partially after the end of the pressure specification so that the patient is allowed expiration against a dynamically regulated counter-pressure, as a function of the flow of the expiration, a therapeutically effective resistance being generated during the exhalation by the regulated partial opening or closing of the valve, which keeps the small airways open for as long as possible and therefore allows comprehensive exhalation of CO2.

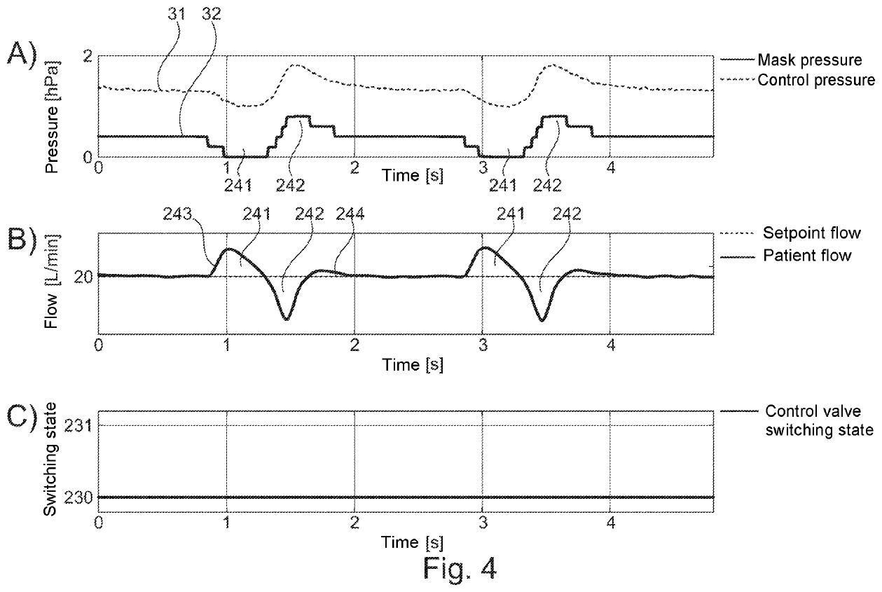

FIG. 4 shows A) the pressure, B) the flow and C) the switching state of the valve 17 for the HFT ventilation (or HFT mode). The HFT ventilation is carried out with a consistent high flow of (warmed and humidified) breathing gas, which is applied via nasal cannulas into both nostrils of the patient. The high flow flushes the nasal dead space, so that more fresh breathing gas is breathed with the inspiration. The nasal cannulas do not in this case seal tightly with the nasal wall, so that exhalation past the cannulas is possible.

In FIG. 4A), the pressure profile of mask pressure 32 and control pressure 31 for HFT ventilation may be seen. The mask pressure and the control pressure of the valve are plotted in FIG. 4A. It may be seen from the profile of the mask pressure 32 that the mask pressure 32 decreases in the time range beyond second one. This short pressure reduction corresponds to the spontaneous inspiration 241 of the patient. The pressure reduction correlates chronologically with the increase of the patient flow 243 in FIG. 4B).

This breathing signal of the patient may be evaluated by the control as a trigger in order to increase the control pressure.

In FIG. 4B), the setpoint flow 244, which is kept substantially consistently at a pre-adjusted level during the HFT ventilation, may be seen. The control drives the breathing gas source in such a way that the setpoint flow 244 is substantially maintained.

In the course of the inspiration 241 of the patient, however, the patient flow 243 increases since the patient actively inhales breathing gas. In this period of time, the patient flow 243 exceeds the setpoint flow 244. In the scope of the subsequent expiration 242, the patient flow 243 falls below the setpoint flow 244. This happens since the active expiration of the patient flows out from the nose, past the nasal cannulas, against the conveyed breathing gas.

The valve 17 remains in a closed state 230 during the HFT ventilation since breathing gas is not intended to escape progressively from the valve but is continuously conveyed during inspiration and expiration to the nasal cannula. For this purpose, the control pressure 31 for the valve 17 is always kept above the mask pressure. This ensures that the patient valve is always closed 230 and no breathing gas is lost through the patient valve. As may be seen from the graph of FIG. 4, it is however possible to recognize the breathing phase, for example in order to determine and display the respiratory rate or inspiration duration.

For example, the control unit is designed and configured to control the breathing gas source in such a way that the mask pressure (or, here, the pressure in the region of the nasal cannulas) decreases during the inspiration. At the end of the inspiration, the mask pressure increases again.

For example, the control unit is designed and configured to control the breathing gas source in such a way that the specified setpoint flow remains below the patient flow during the inspiration. For example, the control unit is designed and configured to control the breathing gas source in such a way that the patient flow increases at the start of the inspiration and the mask pressure furthermore decreases at the start of the inspiration. The patient flow in this case increases to a maximum value which is greatest before the in the middle of the inspiration or in the middle of the inspiration. For example, the specified setpoint flow is in this case initially exceeded slightly, in the range of 5-30% or more than 7%.

For example, the control unit is designed and configured to control the breathing gas source in such a way that the mask pressure increases during the expiration to a maximum pressure, which has decreased again at the end of an expiration.

For example, the control unit is designed and configured to control the breathing gas source in such a way that the mask pressure has an increasing profile during the expiration.

For example, the control unit is designed and configured to control the breathing gas source in such a way that the patient flow decreases at the start during the expiration, and then increases again. The control unit designed and configured to control the breathing gas source in such a way that the patient flow decreases at the start during the expiration, while the mask pressure simultaneously increases.

1.-47. (canceled)

48. An apparatus for breathing gas supply, wherein the apparatus comprises a breathing gas source, a control unit, a memory, a pressure sensor instrument and/or a flow sensor instrument, a replaceable breathing gas tube, at least one connection fitting for the breathing gas tube, a patient interface and a patient valve, wherein the control unit activates a first mode of ventilation for a first period of time, and in this case drives the breathing gas source in order to specify a varying breathing gas parameter for the ventilation, and wherein the control unit activates a further therapy mode for a second period of time, and in this case drives the breathing gas source in order to specify a breathing gas parameter specific for the further therapy mode, the breathing gas tube remaining on the apparatus during the change from the first mode of ventilation to the further therapy mode, and the patient valve being switched for the further therapy mode by the control unit.

49. The apparatus of claim 48, wherein the control unit activates a CPAP mode or an MPV mode or an HFT mode as a further therapy mode and wherein the breathing gas tube is a single tube valve system.

50. The apparatus of claim 48, wherein the control unit activates the further therapy mode, and in this case drives the breathing gas source in order to specify a constant breathing gas parameter independent of or dependent on a breathing phase.

51. The apparatus of claim 48, wherein the further therapy mode is a constant CPAP pressure, which is maintained independently of a breathing phase.

52. The apparatus of claim 48, wherein the patient valve is opened or closed as a function of a breathing phase, the patient valve being closed during the inspiration, and being driven in a controlled way, and temporarily opened in order to ensure exhalation, during the expiration and being operated electrically.

53. The apparatus of claim 48, wherein patient breathing is identified by the control unit from a profile of a flow signal of the flow sensor instrument, and the patient valve is actuated as a function of the flow signal (as a trigger).

54. The apparatus of claim 48, wherein the control unit drives the breathing gas source in order to deliver breathing gas, in order to ensure maintenance of a CPAP pressure level during switching processes of the patient valve.

55. The apparatus of claim 48, wherein the control unit lowers a CPAP pressure at least temporarily when patient breathing is identified by the control unit as expiration from a profile of a flow signal of the flow sensor instrument, the control unit raising the CPAP pressure at least temporarily (pursed lip breathing) when the patient breathing is identified by the control unit as expiration from the profile of the flow signal of the flow sensor instrument.

56. The apparatus of claim 48, wherein the control unit may specify a CPAP pressure at pressure values below 4 hPa, since purging of CO2 from exhaled air by the patient valve takes place reliably even at low pressures.

57. The apparatus of claim 48, wherein the control unit activates a further CPAP therapy mode in response to user selection or automatically, and in this case drives the breathing gas source in order to specify a CPAP pressure, the breathing gas tube remaining at the fitting on the apparatus during a change from the first mode of ventilation to the further therapy mode of CPAP, and the patient valve being closed during inspiration, and being driven in a controlled way, and temporarily opened in order to ensure exhalation, during expiration, patient breathing being identified by the control unit from a profile of a flow signal of the flow sensor instrument, and the patient valve being actuated as a function of the flow signal (as a trigger), the breathing gas source being driven in order to ensure maintenance of the CPAP pressure level during switching processes of the patient valve, the CPAP pressure being specifiable at pressure values below 4 hPa.

58. The apparatus of claim 48, wherein the further therapy mode is a substantially constant breathing gas flow (HFT), which is maintained independently of a breathing phase, the control unit driving the breathing gas source in order to specify a substantially constant breathing gas flow and switching the patient valve into a permanently closed position.

59. The apparatus of claim 58, wherein the control unit is designed and configured to control the breathing gas source for the HFT mode in such a way that a patient flow decreases at a start during expiration, while a mask pressure simultaneously increases.

60. The apparatus of claim 48, wherein the breathing gas tube has a patient valve and remains on the apparatus during the change from ventilation to HFT, and for the HFT the patient valve is closed by a control pressure being sent by the breathing gas source through a pressure tube to the patient valve and/or a humidifier and a heater are activated.

61. The apparatus of claim 48, wherein the control unit specifies a HFT mode with a consistent high flow of breathing gas, which is applied via the patient interface into both nostrils of the patient in such a way that the flow flushes a nasal dead space, the patient interface in this case not being sealing tightly with a nasal wall so that exhalation past the patient interface is possible, a setpoint flow during HFT ventilation being kept substantially consistent at a pre-adjusted level, the patient valve being kept in a closed state during the HFT ventilation since breathing gas is not intended to escape progressively from the patient valve but is continuously conveyed during inspiration and expiration to the patient interface.

62. The apparatus of claim 48, wherein the further therapy mode is an MPV mode, which delivers a breathing gas volume or a breathing gas flow or a pressurized breathing gas to a patient for inspiration as required.

63. The apparatus of claim 48, wherein the control unit drives the breathing gas source in order to specify a breathing gas flow or breathing gas volume or a pressurized breathing gas for inspiration and switches the patient valve into a permanently closed position.

64. The apparatus of claim 48, wherein a breathing effort (inspiration attempt) of a patient is identified by the control unit from a profile of a flow signal or of a pressure signal, and the control unit drives the breathing gas source specifying a breathing gas flow or breathing gas volume when an inspiration attempt by the patient is identified from a profile of the flow signal 8 or of the pressure signal.

65. The apparatus of claim 48, wherein a pressure of a breathing assistance and a volume are adjustable, or wherein the pressure of the breathing assistance and an inspiration time Ti are adjustable.

66. The apparatus of claim 48, wherein limit values are stored or adjustable for a flow signal/or pressure signal, the limit values being a trigger sensitivity and the trigger sensitivity being adjustable.

67. The apparatus of claim 48, wherein a trigger blocking time (in a range of from 0.1 to 10 seconds) can be specified, breathing efforts by a patient, registered by sensing, being ignored by the control unit for a duration of the trigger blocking time.

68. The apparatus of claim 48, wherein an MPV interface (mouthpiece), which is configured in such a way that it is inserted at least partially into a mouth, is used as the patient interface for an MPV mode, the control unit being designed and configured to control the breathing gas source in such a way that a mask pressure has an increasing profile during inspiration and the mask pressure falls more slowly than a setpoint pressure during the expiration.

69. The apparatus of claim 68, wherein the patient valve is briefly opened for an expiration so that a pressure at the mouthpiece is lowered, and the patient valve is subsequently closed.

70. The apparatus of claim 48, wherein the further therapy mode is an MPV mode, which delivers breathing gas to a patient for inspiration as required, a breathing gas pressure being adjustable and/or a breathing gas volume or an inspiration time Ti furthermore being specified, a mouthpiece being used as the patient interface and breathing signals of the patient being registered by sensing when the mouthpiece is in a mouth as a pressure trigger and/or flow trigger in order to start MPV ventilation, the patient being able to keep the mouthpiece in its mouth for expiration, and the control unit then at least temporarily opening the patient valve for the expiration so that the patient can exhale its expiratory air through the fully or partially opened patient valve to the environment, the control unit activating the breathing gas source during the expiration in order to specify a flushing flow to assist flushing of exhaled air out from the tube.

71. The apparatus of claim 48, wherein the further therapy mode is an MPV mode, which temporarily sends a pressurized breathing gas flow to a patient, the apparatus consisting of the following: a breathing gas source, which temporarily sends a pressurized breathing gas flow to airways; a patient interface in the form of a mouthpiece, which can be at least partially inserted into an airway opening of the patient and removed again, the patient interface furthermore being configured so that the breathing gas flow is sent into airways of the patient; at least one sensor, which generates output signals that, when the mouthpiece is at least partially inserted into an airway opening of the patient, indicate that the patient is ready to receive a breathing gas flow through the mouthpiece, the sensor being designed to establish whether the patient has performed breathing efforts; and at least one control unit, which analyzes the sensor signal in order to ascertain whether the patient has performed a breathing effort that exceeds or falls below a limit value for initiating the sending of a temporary pressurized breathing gas flow, the control unit activating the breathing gas source when the limit value for initiating the sending of a temporary pressurized breathing gas flow is reached or exceeded, before specifying a temporary pressurized breathing gas flow, the control unit then activating the temporary pressurized breathing gas flow for an inspiration of the patient.

72. The apparatus of claim 48, wherein the control unit drives the breathing gas source in order to specify a tidal volume for the mode of ventilation.

73. The apparatus of claim 48, wherein the apparatus comprises a compressed gas source and at least one pressure tube, which sends a control pressure to the patient valve.

74. The apparatus of claim 48, wherein the breathing gas tube is a double tube system having an assigned patient valve, the patient valve being located next to the fitting in an apparatus housing.

75. The apparatus of claim 48, wherein the patient valve is configured to be removable from a compartment of a housing, the patient valve comprising a membrane to which a control pressure can be applied in order to block or release a breathing gas flow through the patient valve, the control pressure being generated by the breathing gas source and sent to the patient valve through a control tube.

76. The apparatus of claim 48, wherein the control unit drives the breathing gas source during daytime in order to specify a breathing gas flow and during nighttime in order to specify a varying breathing gas pressure.
